(19) United States
(12) Patent Application Publication
(10) Pub. No.: US 2010/0163461 A1
(43) Pub. Date: Jul. 1, 2010

(54) METHOD AND SYSTEM FOR CONTROLLING THE AMOUNT OF ANTI-FOULING ADDITIVE FOR PARTICULATE-INDUCED FOULING MITIGATION IN REFINING OPERATIONS

(76) Inventors: Chris A Wright, Bordentown, NJ (US); Glen B. Brons, Phillipsburg, NJ (US); Manuel S. Alvarez, Warrenton, VA (US); Peter W. Jacobs, Bound Brook, NJ (US); Sharon A. Feiller, Allentown, PA (US); George A. Lutz, Brick, NJ (US)

Correspondence Address: EXXONMOBIL RESEARCH AND ENGINEERING COMPANY;(formerly Exxon Research and Engineering Company), P.O Box 900, Annandale, NJ, 08801-0900, US

(21) Appl. No.: 12/574,294

(22) Filed: Oct. 6, 2009

Related U.S. Application Data
(60) Provisional application No. 61/136,855, filed on Oct. 9, 2008.

(51) Int. Cl. C10G 75/04; C10G 19/00; G01N 33/00

(52) U.S. Cl. 208/48 AA; 196/46; 73/61.41

(57) ABSTRACT

A method and system for controlling fouling in a hydrocarbon refining process that includes measuring a level of a particulate in a process stream of the hydrocarbon refining process in communication with a hydrocarbon refinery component, identifying an effective amount of additive capable of reducing particulate-induced fouling based at least in part on the measured level of the particulate in the process stream, and introducing the effective amount of additive to the hydrocarbon refining process.

### CROSS REFERENCE TO RELATED APPLICATION

This application relates and claims priority to U.S. Provisional Patent Application No. 61/136,855, filed on Oct. 9, 2008 entitled “Method and System for Controlling the Amount of Anti-Fouling Additive for Particulate-Induced Fouling Mitigation in Refining Operations.”

### FIELD OF THE INVENTION

The present invention relates to methods and systems of controlling the amount of anti-fouling additive to be introduced in an oil refining process.

### BACKGROUND OF THE INVENTION

Petroleum refineries incur additional energy costs, perhaps billions of dollars per year, due to fouling and the resulting attendant inefficiencies caused by the fouling. More particularly, thermal processing of crude oils, blends and fractions in heat transfer equipment, such as heat exchangers, is hampered by the deposition of insoluble asphaltenes and other contaminants (i.e., particulates, salts, etc.). Further, the asphaltenes and other organics are known to thermally degrade to coke when exposed to high heater tube surface temperatures.

For example, fouling in heat exchangers receiving petroleum-type process streams can result from a number of mechanisms including chemical reactions, corrosion, deposit of existing insoluble impurities in the stream, and deposit of materials rendered insoluble by the temperature difference (ΔT) between the process stream and the heat exchanger wall. Naturally-occurring asphaltenes may precipitate from the crude oil process stream, thermally degrade to form a coke and adhere to the hot surfaces. Further, the high ΔT inherent in a heat transfer operation results in high surface or skin temperatures when the process stream is introduced to the heater tube surfaces, which contributes to the precipitation of insoluble particulates. Another common cause of fouling is attributable to the presence of salts, particulates and impurities (e.g. inorganic contaminants) found in the crude oil stream. Iron oxide, iron sulfide, calcium carbonate, silica, sodium chloride, calcium chloride and other solids have all been found to attach directly to the surface of a fouled heater rod and throughout the coke deposit.

The buildup of insoluble deposits in heat transfer equipment creates an unwanted insulating effect and reduces heat transfer efficiency. Fouling also reduces the cross-sectional area of process equipment, which decreases flow rates and desired pressure differentials and reduces process efficiency. To overcome these disadvantages, heat transfer equipment must be taken offline and cleaned mechanically or chemically cleaned, resulting in lost production time.

Accordingly, there is a need to reduce precipitation/adherence of particulates and asphaltenes from the heated surface to prevent fouling, and before the asphaltenes are thermally degraded or coked. This will improve the performance of the heat transfer equipment, decrease or eliminate scheduled outages for fouling mitigation efforts, and reduce energy costs associated with the processing activity.

Various methods have been developed to reduce fouling, including particulate-induced fouling. For example, it has been found that blending a base crude oil with an amount of high solvency dispersive power (HSDP) crude is effective in mitigating fouling. See, e.g., International Application No. PCT/U.S.07/18403 and U.S. patent application Ser. Nos. 11/506,901, 12/222,760, and 12/222,761, each of which is hereby incorporated by reference in its entirety. It has also been found that addition of additives to a process stream is effective in mitigating fouling, particularly particulate-induced fouling. See, e.g. U.S. Provisional Application Nos. 61/136,173 and 61/136,172, each of which is hereby incorporated by reference.

The addition of additives, while of great utility and value for energy savings, does have attendant costs, including the cost of the additive itself and the cost of removing the additive from the process downstream. Accordingly, there is a need to minimize the amount of additive that is introduced to the process in order to achieve the desired reduction in fouling, i.e., using only the required level of additive to achieve the necessary fouling prevention.

### SUMMARY OF THE INVENTION

A method is provided for controlling fouling in a hydrocarbon refining process that includes measuring a level of particulate (e.g. the particulate concentration) in a process stream, including those streams in a hydrocarbon refining process, identifying an effective amount of additive capable of reducing particulate-induced fouling of process equipment in that stream based at least in part on the measured level of the particulate in the process stream, and, introducing and controlling the amount of additive to the hydrocarbon refining process to mitigate the fouling.

In accordance with one aspect of the invention, the effective amount of additive is identified based, at least in part, on a relative fouling potential of the crude oil. A method is provided for determining the relative fouling potential of a crude oil that includes obtaining at least two measurements. The first measurement is a measurement of a characteristic property related to the amount of fouling caused by the crude oil in the absence of any measurable particulate. The second measurement is a measurement of the characteristic property indicative of the amount of fouling caused by the crude oil in the presence of a predetermined amount of particulate. The first and second measurements are then compared to identify the relative fouling potential of the crude oil.

The present application also provides an additive control system for controlling fouling in a hydrocarbon refining system that includes a source of additive capable of reducing particulate-induced fouling in a hydrocarbon refining system, a valve to introduce to a process stream of the hydrocarbon refining system the additive capable of reducing particulate-induced fouling, a measuring device to measure a level of particulate in the process stream of the hydrocarbon refining system, and a controller to control an amount of additive introduced into the process stream via the valve based upon the level of particulate measured in the process stream. The additives are preferably introduced at a strategic location in the process unit to enhance the additive's effectiveness.

### BRIEF DESCRIPTION OF THE DRAWINGS

The application will now be described in conjunction with the accompanying drawings in which:

FIG. 1A is a schematic of an exemplary process scheme demonstrating the communication between particulate measuring device D, controller C and valve V of additive source S.

FIG. 1B demonstrates inputs to the controller which will be inserted into a pre-selected algorithm to determine valve position for a hypothetical distributed control system.

FIG. 1C is a schematic of a hydrocarbon refining system depicting possible locations for the introduction of additive.

FIG. 2 is a schematic of the Alcor Hot Liquid Process Simulator (HPLS) employed in Examples 1 and 2 of this application.

FIG. 3 is a graph demonstrating the reduction in the efficiency of an anti-foulant from 60% fouling reduction to 40% fouling reduction due to an increase in the amount of particulates present.

FIG. 4 is a graph demonstrating the effects of particulates/solids on the fouling of whole crude oil B.

FIG. 5 is a graph demonstrating the effects of particulates/solids on the fouling of whole crude oil C.

### DETAILED DESCRIPTION OF THE INVENTION

### Definitions

The following definitions are provided for purpose of illustration and not limitation.

As used herein, the term “fouling” generally refers to the accumulation of unwanted materials on the surfaces of processing equipment or the like.

As used herein, the term “particulate-induced fouling” generally refers to fouling caused primarily by the presence of organic or inorganic particulates. Organic particulates include, but are not limited to, insoluble matter precipitated out of solution upon changes in process conditions (e.g. temperature, pressure, or concentration changes) or a change in the composition of the feed stream (e.g. changes due to the occurrence of a chemical reaction). Inorganic particulates include, but are not limited to, silica, iron oxide, iron sulfide, alkaline earth metal oxide, sodium chloride, calcium chloride, metal silicates and metal aluminosilicates, magnesium chloride and other inorganic salts. One major source of these particulates results from incomplete removal in the desalting process and/or other particulate removing process.

As used herein, the term “crude hydrocarbon refinery component” generally refers to an apparatus or instrumentality of a process to refine crude hydrocarbons, such as an oil refinery process, which is, or can be, susceptible to fouling. Crude hydrocarbon refinery components include, but are not limited to, heat transfer components such as a heat exchanger, a furnace, a crude preheater, a coker preheater, or any other heaters, a FCC slurry bottom, a debutanizer exchanger/tower, other feed/effluent exchangers and furnace air preheaters in refinery facilities, rotating equipment such as compressor components in refinery facilities and steam cracker/reformer tubes in petrochemical facilities. Crude hydrocarbon refinery components can also include other process equipment in which heat transfer can take place, such as a fractionation or distillation column, a scrubber, a reactor, a liquid-jacketed tank, a pipestill, a coker and a visbreaker. It is understood that “crude hydrocarbon refinery components,” as used herein, encompasses tubes, piping, baffles and other process transport mechanisms that are internal to, at least partially constitute, and/or are in direct contact with the process fluid with, any one of the above-mentioned crude hydrocarbon refinery components.

Reference will now be made to various aspects of the present application in view of the definitions above.

One aspect of the present application provides a method of controlling fouling in a hydrocarbon refining process including measuring a level of a particulate in a process stream of the hydrocarbon refining process in communication with a hydrocarbon refinery component, identifying an effective amount of additive capable of reducing particulate-induced fouling based, at least in part, on the measured level of particulate in the process stream, and introducing the effective amount of additive to the hydrocarbon refining process to mitigate the fouling.

In one embodiment the particulate includes one or more of iron oxide, iron sulfide, calcium carbonate, silica, or, other inorganic salts. In a preferred embodiment, the particulate is iron oxide. In another preferred embodiment, the particulate is iron sulfide. In another preferred embodiment, the inorganic salt is selected from sodium chloride and calcium chloride.

In one embodiment, the effective amount of additive is introduced to the hydrocarbon refining process in real-time either continuously, periodically, or, at varying injection rates based at least in part on a real-time measured level of the particulate in the process stream. Alternatively, the effective amount of additive is introduced to the hydrocarbon refining process based at least in part on the measured level of the particulate in the process stream over a predetermined period. For example, it is generally preferred that the effective amount of additive is determined based on a level of particulate measured over a period of at least 4 hours, or 8 hours, or 12 hours or 24 hours. Alternatively, a new additive dose rate is fixed based on measurements performed when either a process condition change takes place, or, a raw material change takes place. It is more preferred that the effective amount of additive be determined in real time, based on a real time level of measured particulate.

In accordance with another aspect of the invention, the effective amount of additive is identified based at least in part on a relative fouling potential of a crude oil that is present in the process stream in the presence of the particulate. Particularly, and for purpose of illustration and not limitation, the relative fouling potential of the process stream can be measured by obtaining a first measurement of a characteristic indicative of an amount of fouling caused by the crude oil in the absence of any measurable particulate, obtaining a second measurement of the characteristic indicative of an amount of fouling caused by the crude oil in the presence of a predetermined amount of particulate, and comparing the first measurement with the second measurement to identify the relative fouling potential of the crude oil.

In a still further embodiment, the first measurement and the second measurement are normalized based on the heat transfer ability of the crude oil blend. That is, the measurement indicative of fouling is normalized such that various phenomenon besides fouling that can reduce the heat transfer ability of the crude oil blend are not allowed to influence the value that is to be indicative of fouling. For example, environmental conditions (e.g. fluctuating ambient temperatures) could have an impact on the characteristic indicative of an amount of fouling, since a reduction of heat transfer can be attributable to such environmental influences, and not to fouling of heat transfer equipment (e.g. heat exchangers). By normalizing obtained values by the heat transfer ability of the crude oil blend, the effects of fouling are isolated and more suitable for comparison with other normalized values.

In a further embodiment of the present application, the process is repeated for at least two distinct crude oils, and the relative fouling potential for the first crude oil blend is compared to the relative fouling potential for the second crude oil blend. The relative fouling potentials can be used for selecting the crude oil to be used in a hydrocarbon refining process.

In accordance with another aspect of the invention, an additive control system is provided for controlling fouling in a hydrocarbon refining system that includes a source of additive capable of reducing particulate-induced fouling in a hydrocarbon refining system, a valve to introduce to a process stream of the hydrocarbon refining system the additive capable of reducing particulate-induced fouling, a measuring device to measure a level of particulate in the process stream of the hydrocarbon refining system, and a controller to control an amount of additive introduced into the process stream via the valve based upon the level of particulate measured in the process stream. In a preferred embodiment, the additive is introduced into the process in a strategic location and/or a manner that properly disperses the additive to enhance its effectiveness.

In one specific embodiment, the crude hydrocarbon refinery component is selected from a heat exchanger, a furnace, a crude preheater, a coker preheater, a FCC slurry bottom, a debutanizer exchanger, a debutanizer tower, a feed/effluent exchanger, a furnace air preheater, a flare compressor component, a steam cracker, a steam reformer, a distillation column, a fractionation column, a scrubber, a reactor, a liquid-jacketed tank, a pipestill, a coker, and a visbreaker. In a preferred embodiment, the crude hydrocarbon refinery component is a heat exchanger.

Exemplary further embodiments of the present application are provided below for illustrative purposes, and not for purposes of limitation.

### Exemplary Control System of the Present Application

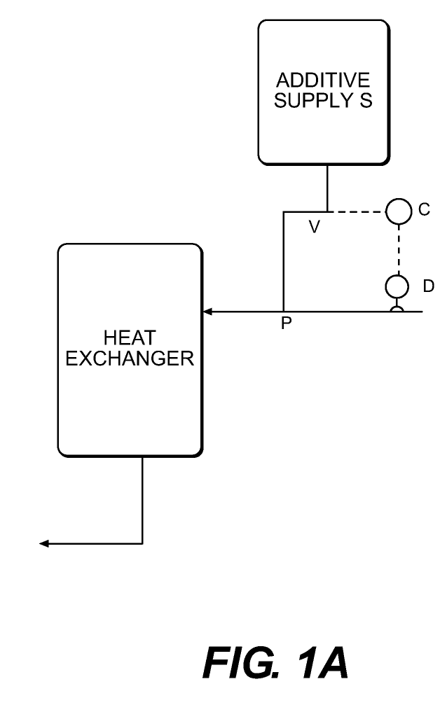

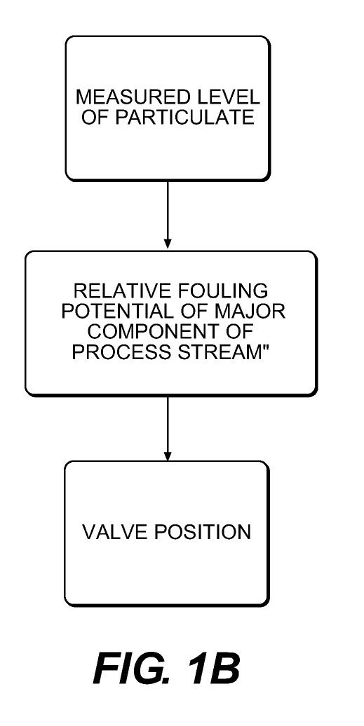

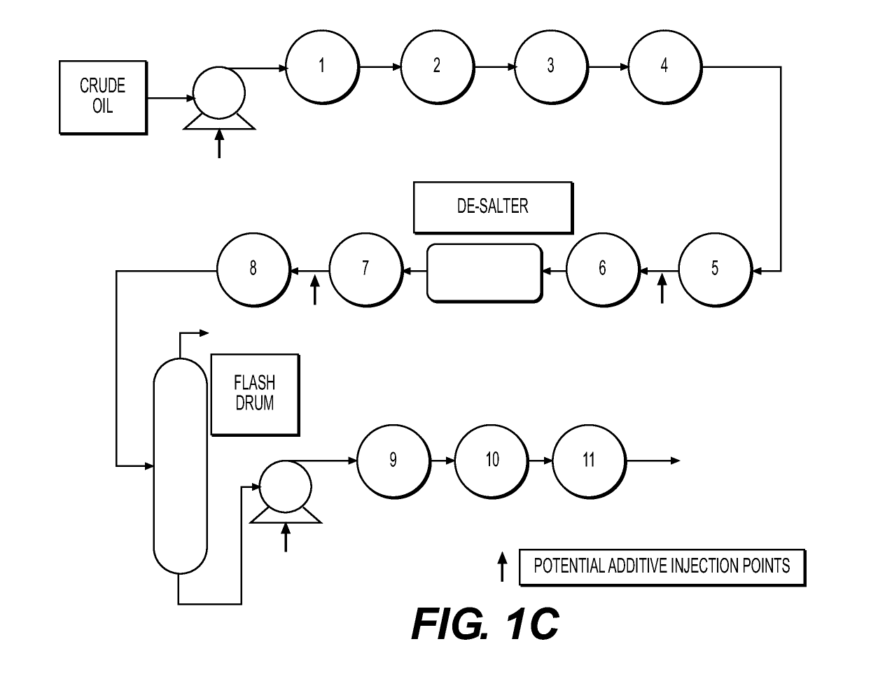

FIG. 1A is a schematic of an exemplary process scheme demonstrating the communication between particulate measuring device D, controller C and valve V of additive source S. The exemplary process scheme includes a source of additive capable of reducing particulate-induced fouling in a hydrocarbon refining system. The additive source is in fluid communication with the process stream of the hydrocarbon refining systems via a valve “V”. As used herein, the valve is defined broadly and can be any suitable mechanism capable of controlling the introduction of additive into the process stream. The additive injection point location into the process is chosen to increase its effectiveness. For example, if the additive that is chosen is one that, due to additive chemistry, requires some time to complete an anti-fouling reaction, the process flow and piping details should be considered to provide an effective application such that adequate residence time is provided.

Controller “C” in FIG. 1A controls valve “V” based on particulate level information obtained from measuring device “D” and/or inputs regarding the relative fouling potential of a crude oil present in the process stream “P”. The controller, measuring device and valve are components in a distributed control system (DCS), and can be modified by one skilled in the art in accordance with the method and system described herein. Distributed control systems are available from, for example, Honeywell International Inc. (Morristown, N.J.); Emerson Process Management division of Emerson Electric Company (St. Louis, Mo.), including Fisher-Rosemount products (Eden Prairie, Minn.); Yokogawa Corporation of America (Newnan, Ga.); and Shinkawa, SEC of America (Ocean Isle Beach, N.C.).

The measuring device “D” measures particulate levels in the process stream “P”. The measuring device “D” is in communication with the Controller “C”, which in turn is in communication with the valve “V” (discussed below). Exemplary devices that can be used in the present application are described, for example, in U.S. Pat. Nos. 4,506,543; 5,121,629; and 3,710,615; each of which are hereby incorporated by reference in their entirety. Suitable measuring devices can be commercially obtained from, for example, Stanhope-Seta (Surrey, UK), Horiba Instruments Inc. (Irvine, Calif.) and Nanosight Ltd. (Salisbury, UK).

In a non-limiting, exemplary embodiment, the measuring device “D” determines the level of calcium, magnesium and/or sodium content in a process stream in a hydrocarbon refining operation. In a second exemplary embodiment the measuring device “D” makes use of on-line video microscopy and suitable particle identification algorithms to determine the amount and/or optical characteristics of the particulates. The specific particulates to be measured by the measuring device can be varied, and is not limited. A person of ordinary skill in the art can select the proper particulate to measure based on the particular refining system and the crude oil composition (e.g. a crude oil blend) propensity to foul in the presence of the specific particulate.

In addition to, or in lieu of receiving the level of particulate in the process stream “P”, the Controller can receive an input based on the propensity of the process stream to foul. The propensity of the process stream “P” to foul helps predict how the process stream will react when processed with the particulate levels measured by the measuring device “D”. For example, it has been found that some crude oil blends are more susceptible to particulate-induced fouling than others. When a crude oil blend having a greater relative fouling potential is used in the refining system, a greater amount of additive will be required to be introduced for a given particulate level, as compared to a crude oil blend previously found to have a low relative fouling potential.

Using a pre-determined algorithm, the controller C will output a signal to Valve V based on one or more of: (a) the measured level of particulate in the process stream and (b) the relative fouling potential of one or more components of the process stream (e.g. the relative fouling potential of a crude oil blend that is the major constituent of the process stream). FIG. 1B depicts exemplary inputs to the controller which will be inserted into the pre-determined algorithm to determine the valve position as part of a hypothetical distributed control system.

Embodiments of the present invention can also employ particulate identification algorithms, which can further assist in determining valve position to provide the desired amount of additive to the refining process. For example, the algorithms and sensors described in U.S. Pat. No. 6,649,416, hereby incorporated by reference in its entirety, can be employed in the methods and systems of the present application.

In one particular embodiment, the controller factors both particulate level and relative fouling potential. Alternatively, the controller can control the valve based on the particulate level alone, or the relative fouling potential alone. The person of ordinary skill in the art can adjust the algorithm so that the relative contribution of each of the two components is best-suited for the particular refining process for which it is applied. For example, when field observations suggest that the control system is not being sufficiently responsive to changes in particulate level for the particular refining system, the relative contribution of the measured particulate level factor can be increased. Similarly, when field conditions suggest that the control system is being overly responsive to slight fluctuations in the measured particulate level for the particular refining system, the relative contribution of the measured level of particulate in the process stream can be reduced.

The Controller “C” is in communication with a valve (flow restrictor) “V”. The valve can be any suitable mechanism that regulates desired amounts or flow rates of additive to be introduced into the process stream. Examples include a ball valve, butterfly valve, gate valve, check valve, quarter turn valve, sanitary valve, solenoid control valve, and any other valve appropriate to control of the flow of additives that reduce particulate-induced fouling depending on the form and typical flow rates of the additive to be introduced. Alternatively, a variable speed metering pump can be used to inject the additive into the process. Where the speed of the pump is controlled based on the measured particulate concentration. Valves can be obtained commercially from, for example, Fischer Process Industries (Suwanee, Ga.); United Valve (Houston, Tex.), and Sulzer Valves (Rancho Santa Margarita, Calif.).

In one embodiment, the measuring device is located immediately upstream from a heat-exchanger, or other crude hydrocarbon refinery component, particularly hydrocarbon refinery components that are susceptible to particulate-induced fouling. Similarly, in one embodiment, the additive is introduced to the process stream immediately upstream from a heat-exchanger, or other crude hydrocarbon refinery component, particularly hydrocarbon refinery component.

Alternatively, the measuring device can be located directly upstream from, or otherwise in close proximity to, other hydrocarbon refinery components such as, but not limited to, a heat exchanger, a furnace, a crude preheater, a coker preheater, a FCC slurry bottom, a debutanizer exchanger, a debutanizer tower, a feed/effluent exchanger, a furnace air preheater, a flare compressor component, a steam cracker, a steam reformer, a distillation column, a fractionation column, a scrubber, a reactor, a liquid-jacketed tank, a pipestill, a coker, and a visbreaker.

Alternatively, the additive can be introduced into an upstream process unit such as a desalter to improve the particulate removal efficiency there, and mitigate the fouling effect of particulate on other equipment downstream by reducing the particulate concentration. For example, water soluble additives can be added upstream of a mixing valve to enhance the operation of a desalting operation.

### Measuring Fouling and Determining Relative Fouling Potential (RFP)

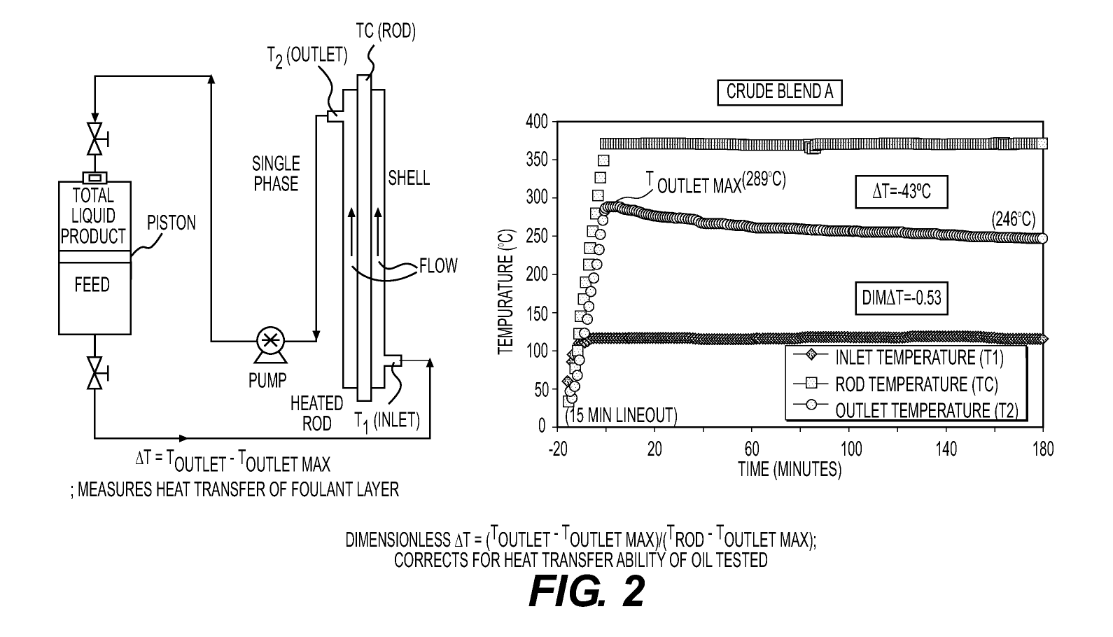

Fouling can be measured, for example, by testing a crude oil blend in an Alcor Hot Liquid Process Simulator (HPLS). An example of such a unit is shown in FIG. 2, and is commercially available from Alcor Petroleum Corporation (Westbury, N.Y.). The device contains a heated rod over which passes a flow of a crude oil blend at a constant inlet temperature. Heat is transferred from the rod (which simulates a heat exchanger) to the crude oil blend, and the temperature of the crude oil blend as it exits the unit is measured. In this non-limiting example, the characteristic indicative of an amount of fouling is the difference in temperature (ΔT) between the outlet crude oil temperature at a preselected time and the maximum outlet crude oil temperature observed at anytime during the trial:

ΔT=(Tₒᵤₜₗₑₜ−T_{outlet max}). Eq. 1

The reduction in temperature, i.e. the reduction in heat transfer from the rod, can be attributed to the fouling that occurs on the rod.

As discussed above, the measurement of a characteristic indicative of an amount of fouling can be normalized based on the heat transfer ability of the crude oil tested. For example, with reference to FIG. 2 and the above-described Alcor Hot Liquid Process Simulator (HPLS), the following “dimensionless ΔT” or “dimΔT” can be determined as shown below:

dimΔT=(Tₒᵤₜₗₑₜ−T_{outlet max})/(T_{rod}−T_{outlet max}). Eq. 2

The denominator accounts for the heat transfer ability of the oil tested. The dimΔT is a non-limiting example of a characteristic indicative of an amount of fouling that has been normalized based on the heat transfer ability of the crude oil.

A first measurement of fouling can be made using Alcor Hot Liquid Process Simulator (HPLS) in the absence of a particulate, and compared to a second measurement of fouling using the Alcor Hot Liquid Process Simulator (HPLS) in the presence of a pre-selected amount of particulate. Comparison of these two measurements provides the relative fouling potential of the crude oil blend. One such means of quantifying the relative fouling potential for a given oil is shown below, where dimΔT is the fouling measurement for a crude oil “A” in the absence of a particulate and dimΔT₂₀₀ is a fouling measurement for a crude oil “A” in the presence of 200 ppm of a given particulate:

RFP_{A}=(dimΔT₂₀₀−dimΔT)/(dimΔT). Eq. 3

It is noted that Equations 1-3 and the above description is provided by way of example; the methods and systems of the present invention are not limited to the particular algorithms and equations described herein. In various embodiments the algorithms employed are normalized to provide a unit measure of fouling, as opposed to an absolute value.

Alternatively, fouling can be measured based on its the fouling rate as compared to a “standard fouling rate” (e.g., a multiple or fraction of the standard fouling rate). The standard fouling rate is a unit amount of fouling measured using a particular fluid (e.g., a specific, defined type of crude oil), run at specified, constant conditions for a specified period of time in a specified apparatus. The fouling rate can be measured in the presence and absence of a particular amount of particulate respectively.

A person of ordinary skill in the art can develop other techniques and devices for measuring fouling and quantifying the fouling shown in the presence and absence of a particulate. For example, alternative methods of measuring fouling include, but are not limited to, measurements obtained from microscopes (including video microscopes) based on, for example, the visual observation of material accumulating on the surface. Microscopes can be commercially obtained from, for example, Olympus Corporation (Center Valley, Pa.) and YSC Technologies (Fremont, Calif.).

Fouling also can be ascertained by measuring the mass of material deposited on a surface or by profilometry or measuring the thickness of the deposit on a surface. Measurement of the ash content of said deposited material can indicate the presence or absence of inorganic particulates, as disclosed, for example in commonly assigned co-pending U.S. patent application Ser. No. 11/173,979 (Publication No. US 2006/0014296), which is hereby incorporated by reference in its entirety. Measurement of the atomic H:C ratio of the deposited material can indicate the presence or absence of organic particulate contaminants as disclosed, for example in commonly assigned co-pending U.S. patent application Ser. No. 11/173,979 (Publication No. US 2006/0014296), which is hereby incorporated by reference in its entirety. Alternatively, the pressure drop or flow resistance across a heat exchanger or other crude hydrocarbon refinery component can be measured, such as by measuring the pressure drop at a small orifice in close proximity to the crude hydrocarbon refinery component, and/or by measuring frequency shifts of a resonator near the crude hydrocarbon refinery component as disclosed, for example in commonly assigned co-pending U.S. patent application Ser. No. 11/710,657 (Publication No. US 2007/0199379), which is hereby incorporated by reference in its entirety. Fouling can also be measured using a high temperature fouling unit (HTFU).

Further, in addition to the above-described Alcor HPLS, other devices, which optionally employ one or more of the above-described methods of measuring fouling, can be selected by a person of ordinary skill in the art. For example, coupons or plates in an autoclave or draft-tube autoclave devices can be employed, such as the autoclave device described in Example 6 of International Publication No. WO 2005/113726, which is hereby incorporated by reference. Other devices that can be used in accordance with the methods and systems of the present application include organic deposition units, and those devices disclosed in Chapter 8 of the Heat Exchanger Design Handbook by T. Kuppan, CRC Press (2000), which is hereby incorporated by reference in its entirety. It is understood, however, that present application is not limited to the devices and methods disclosed herein to measure fouling,

### Additives of the Present Application

The additives of the present application are generally soluble in a typical hydrocarbon refinery stream and can thus be added directly to the process stream, alone or in combination with other additives that contribute to either reduce fouling or improve some other process parameter in order to enhance the refining process.

The method and system described herein can be used with any suitable additives capable of reducing particulate-induced fouling in hydrocarbon refining systems. For purposes of illustration and not limitation, examples of suitable additives include polyalkyl succinic acid derivatives, including boron-modified polyalkyl succinic acid derivatives such as those additives described in U.S. Ser. No. 61/136,172; and metal sulfonate additives, such as those described in U.S. Ser. No. 61/136,173. Each of these applications is hereby incorporated by reference in their entirety.

One embodiment of the present application provides a method of choosing an appropriate additive based on the relative fouling potential of the crude oil or crude oil blend employed in the process. For example, if the relative fouling potential of the crude oil is particularly high, then process economics may justify the use of a higher-priced additive. Alternatively, if the relative fouling potential of the crude oil or crude oil blend is relatively low, then a lower-priced additive can be employed. Information about the susceptibility of the crude oil to fouling thus can be used in the selection of a particular additive for a refining process in which the crude oil is a major component.

The additives of the present application can be provided in a solid (e.g. powder or granules) or preferably in a liquid form directly to the process stream. As noted below, the additives can be added alone, or combined with other components to form a composition for reducing fouling (e.g. particulate-induced fouling). Any suitable technique and mechanism can be used for introducing the additive to the process stream, as known by a person of ordinary skill in the art in view of the process to which it is employed.

The additives of the present application are provided in compositions that prevent fouling, including particulate-induced fouling. In addition to the additives of the present application, the compositions can optionally further contain a hydrophobic oil solubilizer for the additive and/or a dispersant for the additive.

Suitable solubilizers can include, for example, surfactants, carboxylic acid solubilizers, such as the nitrogen-containing phosphorous-free carboxylic solubilizers disclosed in U.S. Pat. No. 4,368,133, hereby incorporated by reference in its entirety.

Also as disclosed in U.S. Pat. No. 4,368,133, hereby incorporated by reference, surfactants that can be included in compositions of the present application can include, for example, any one of a cationic, anionic, nonionic or amphoteric type of surfactant. See, for example, McCutcheon's “Detergents and Emulsifiers”, 1978, North American Edition, published by McCutcheon's Division, MC Publishing Corporation, Glen Rock, N.J., U.S.A., including pages 17-33, which is hereby incorporated by reference in its entirety.

The compositions of the present application can further optionally include, for example, viscosity index improvers, anti-foamants, antiwear agents, demulsifiers, anti-oxidants, and other corrosion inhibitors. It is noted that water may have a negative impact on boron-containing additives. Accordingly, it is advisable to add boron-containing additives at process locations that have a minimal amount of water.

Furthermore, the additives of the present application can be added with other compatible components that address other problems that may present themselves in a oil refining process known to one of ordinary skill in the art.

### EXAMPLES

The present application is further described by means of the examples, presented below. The use of such examples is illustrative only and in no way limits the scope and meaning of the invention or of any exemplified term. Likewise, the invention is not limited to any particular preferred embodiments described herein. Indeed, many modifications and variations of the invention will be apparent to those skilled in the art upon reading this specification. The invention is therefore to be limited only by the terms of the appended claims along with the full scope of equivalents to which the claims are entitled.

### Example 1

FIG. 2 shows the Alcor testing configuration used for measuring the relative fouling provided by a given crude oil in a simulated heat exchanger. The testing arrangement includes a reservoir containing a feed supply of crude oil. The feed supply is heated to a selected temperature (e.g. 150° C./302° F.). The housing shell contains a vertically oriented heated rod. The heated rod is typically formed from a carbon steel. The heated rod simulates a tube in a heat exchanger. The heated rod is electrically heated to a preset temperature (e.g. 370° C./698° F.) and maintained at such temperature during the trial. The feed supply is pumped across the heated rod at a constant flow rate (e.g. 3.0 mL/minute). The spent feed supply is collected in the top section of the reservoir. The spent feed supply is separated from the untreated feed supply oil by a sealed piston, thereby allowing for once-through operation. The system is pressurized with nitrogen (e.g. 400-500 psig) to ensure gases remain dissolved in the oil during the test. Thermocouple readings are recorded for the bulk fluid inlet and outlet temperatures and for surface of the rod.

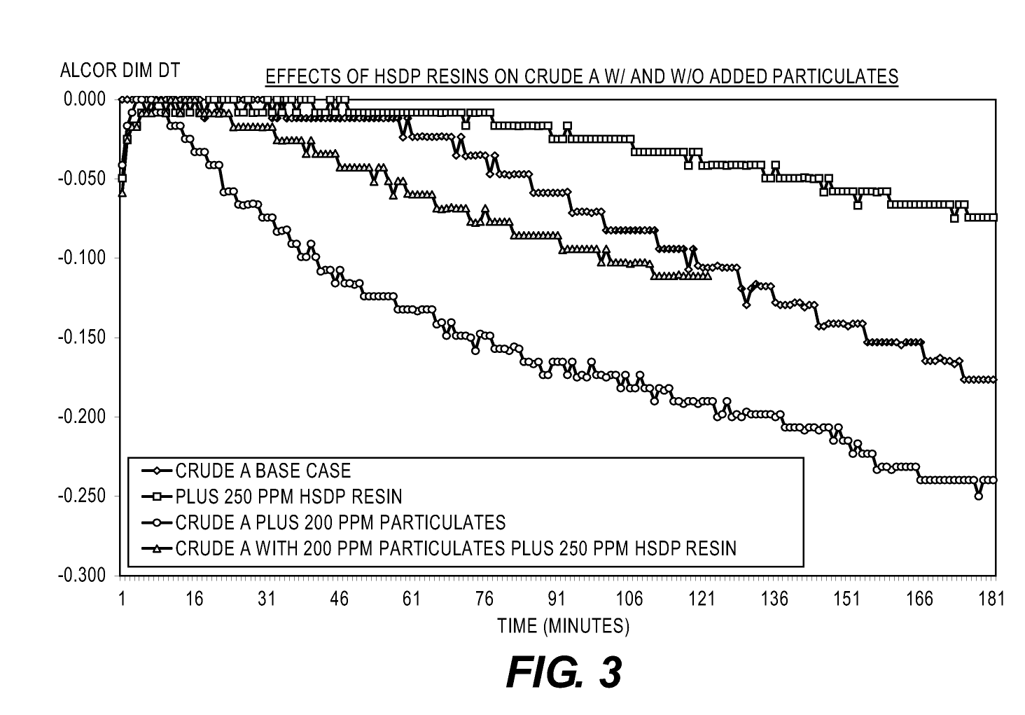

Crude A containing 300 wppm of native iron oxide particulates (measured as filterable solids) was measured. There is a fouling reduction of about 60% upon the addition of 250 wppm of an HSDP anti-fouling resin. However, when the particulates level is increased by further addition of 200 wppm of iron oxide to the fouling crude oil blend, the fouling reduction upon the addition of the same 250 wppm of the same HSDP anti-fouling resin is only about 40%. If an online monitoring system is in place, the spike of additional particulate matter will be observed and therefore additional antifouling additive can be used to maintain the more preferable 60% reduction in fouling levels. The results are shown in FIG. 3.

### Example 2

During the constant surface temperature testing, foulant forms, deposits and builds up on the heated surface. The organic portion of the foulant deposits thermally degrade to coke. The coke deposits cause an insulating effect that reduces the efficiency and/or ability of the surface to heat the oil passing over it. The resulting reduction in outlet bulk fluid temperature continues over time as fouling continues. This reduction in temperature can be referred to as the outlet liquid ΔT or dT and can be dependent on the type of crude oil/blend, testing conditions and/or other effects, such as the presence of salts, sediment or other fouling promoting materials. Typically, the Alcor fouling test is carried out for 180 minutes. The total fouling, as measured by the total reduction in outlet liquid temperature is referred to as ΔT180 or dT180.

Alcor Dimensionless Delta T (DimΔT or Dim dT). The Alcor fouling test simulations provide a measurement of heat transfer resistance due to foulant deposition. A simple measure of this resistance can be obtained from the oil outlet temperature, noted as Tₒᵤₜₗₑₜ in FIG. 2. In the example Alcor run plotted in FIG. 2, the ΔT180 value was found to be −43° C. This value is negative and reflects that the foulant layer deposited on the constant temperature rod after the 180 minute test. The ΔT value provides a simple way of comparing differences in relative heat transfer resistance caused by different oils. For example, a small negative value indicates less deposit formed and lower fouling, while a large negative value indicates that more deposit formed and higher fouling.

When making relative comparisons of different oils, the heat transfer characteristics (viscosity, density, heat capacity, etc.) of the oils being tested should be taken into consideration. This is because oils with higher heat capacities can lead to higher maximum oil outlet temperatures during testing. In cases with added solids/particulates, the concentration of suspended solids can impact heat transfer and affect the maximum oil outlet temperatures. Besides fouling, environmental conditions (e.g., fluctuating ambient temperatures) can also impact the maximum oil outlet temperatures achieved. By correcting for these different heat transfer impacts, relative rankings between different oils and different test runs can be carried out more consistently. This correction is achieved by dividing the ΔT, as described above, by a measure of heat transferred from the rod during each experiment, which is simply the rod temperature minus maximum outlet temperature, shown in the Equation below:

dimΔT=(T_{OUTLET}−T_{OUTLETMAX})/(T_{ROD}−T_{OUTLETMAX}) Eq. 2

Because the final value is unit-less, it is referred to as dimensionless ΔT or “dimΔT” and can also be referred to as the Fouling Potential (FP). For example shown in FIG. 2, the dimΔT180 value is calculated to be −0.53. The FP value that would be noted for this example is 0.53.

The Fouling Potential (FP) factors for whole crude oils and blends need to include the effects that particulates have on fouling of the hydrocarbon refining system. Some crudes have been shown to be more sensitive than others in how they are affected by the presence of particulates/solids. The examples below are provided to demonstrate this “sensitivity” and support the need for testing with and without the solids. A few crude oils have also been shown to exhibit no fouling until particulates are present.

The FP factors are noted as their final Alcor Dim dT after 180 minutes. The FP factor with added particulates are noted as FP₂₀₀ and reflect the final Alcor Dim dT after 180 minutes and reflect the sensitivity of the fouling of the whole crude oil to the 200 ppm solids.

The examples described below show the Alcor fouling data from two crude oils (one moderate-fouling, one non-fouling) that were filtered to remove native particulates. Results are also included to demonstrate the effects of including 200 ppm particulates (inorganic). In each case, the fouling is increased significantly.

Crude B (moderate fouling) FP=0.24 FP₂₀₀=0.36

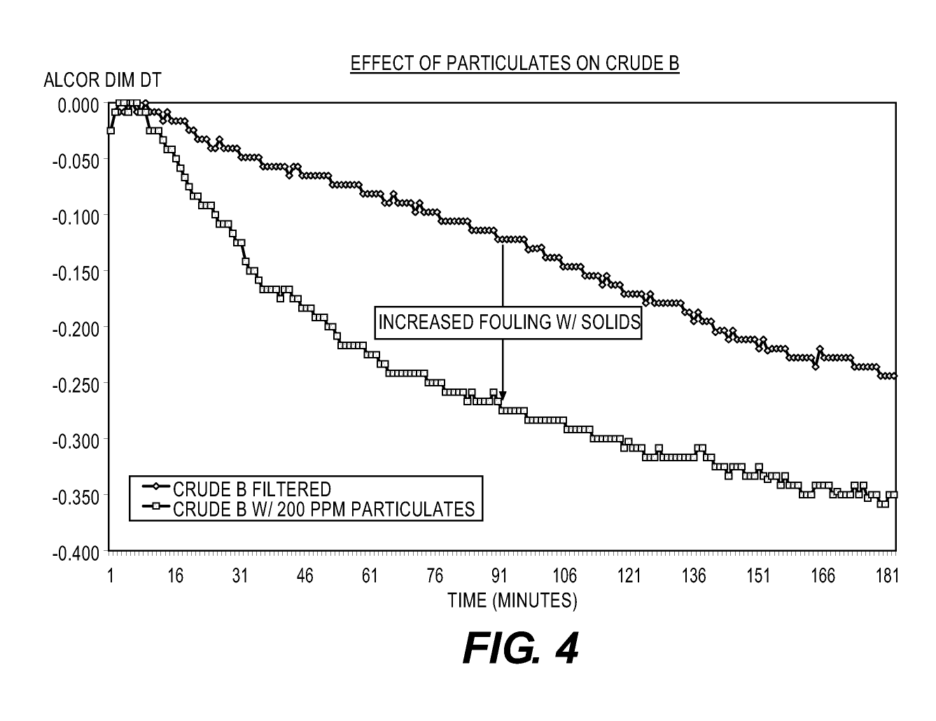

Note that the fouling is increased by 50% with particulates present and the Relative Fouling Potential can be quantified as 0.5. The results are shown in FIG. 4.

Crude C (high fouling): FP=0 FP₂₀₀=0.33

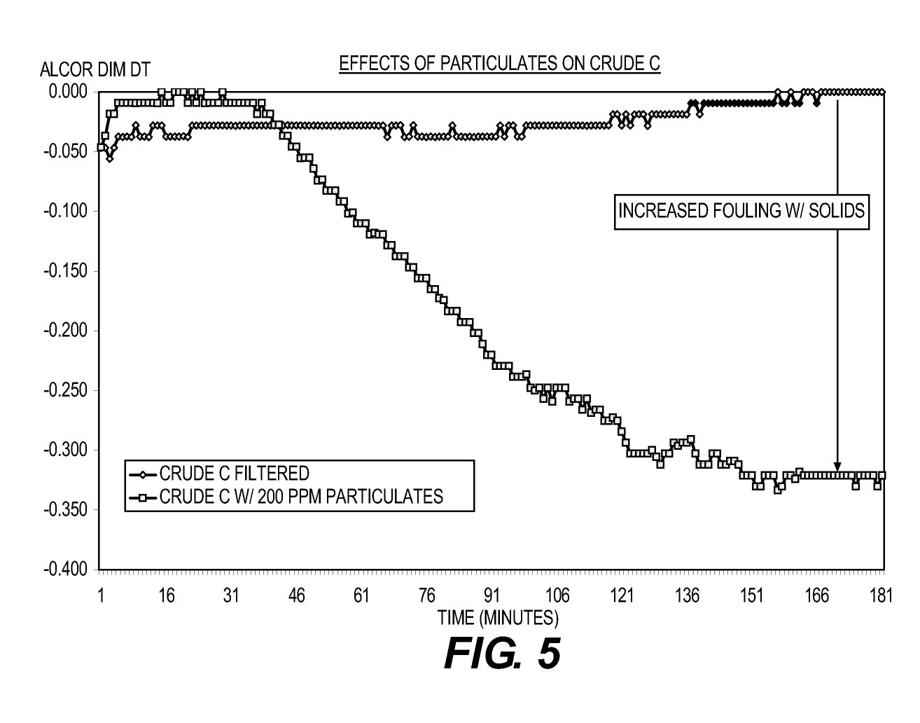

Note that the fouling is increased from zero to high-fouling after particulates were introduced. The results are shown in FIG. 5.

Hence, the presence of particulate for Crude C has a much more drastic effect on fouling, as compared to Crude B.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are approximate, and are provided for description.

Patents, patent applications, publications, product descriptions, and protocols are cited throughout this application, the disclosures of each of which is incorporated herein by reference in its entirety for all purposes.

1. A method of controlling fouling in a hydrocarbon refining process comprising:
(a) measuring a level of a particulate in a process stream of the hydrocarbon refining process in communication with a hydrocarbon refinery component;
(b) identifying an effective amount of additive capable of reducing particulate-induced fouling based at least in part on the measured level of the particulate in the process stream; and
(c) introducing the effective amount of additive to the hydrocarbon refining process.

2. The method of claim 1, wherein the effective amount of additive is introduced to the hydrocarbon refining process in real-time based at least in part on a real-time measured level of the particulate in the process stream.

3. The method of claim 1, wherein the effective amount of additive is introduced to the hydrocarbon refining process based at least in part on the measured level of the particulate in the process stream over a predetermined period.

4. The method of claim 3, wherein the predetermined period is at least four hours.

5. The method of claim 3, wherein the predetermined period is at least eight hours.

6. The method of claim 1, wherein the effective amount of additive is identified based at least in part on a relative fouling potential of a crude oil present in the process stream in the presence of the particulate.

7. The method of claim 6, wherein the relative fouling potential is determined by a method comprising:
(a) obtaining a first measurement of a characteristic indicative of an amount of fouling caused by the crude oil in the absence of any measurable particulate;
(b) obtaining a second measurement of the characteristic indicative of an amount of fouling caused by the crude oil in the presence of a predetermined amount of particulate;
(c) comparing the first measurement and the second measurement to identify the relative fouling potential of the crude oil.

8. The method of claim 7, wherein the first measurement and the second measurement are normalized based on the heat transfer ability of the crude oil.

9. The method of claim 7, wherein the first and second measurements of a characteristic indicative of an amount of fouling are determined on an Alcor Hot Liquid Process Simulator.

10. The method of claim 7, wherein the crude hydrocarbon refinery component is selected from a heat exchanger, a furnace, a crude preheater, a coker preheater, a FCC slurry bottom, a debutanizer exchanger, a debutanizer tower, a feed/effluent exchanger, a furnace air preheater, a flare compressor component, a steam cracker, a steam reformer, a distillation column, a fractionation column, a scrubber, a reactor, a liquid-jacketed tank, a pipestill, a coker, and a visbreaker.

11. The method of claim 10, wherein the crude hydrocarbon refinery component is a heat exchanger.

12. The method of claim 10, wherein the particulate comprises one or more of iron oxide, iron sulfide, calcium carbonate, metal silicate, metal aluminosilicate, silica, or an inorganic salt.

13. The method of claim 12, wherein the particulate is iron oxide.

14. The method of claim 12, wherein the particulate is iron sulfide.

15. The method of claim 12, wherein the inorganic salt is selected from sodium chloride and calcium chloride.

16. A method of controlling fouling in a hydrocarbon refining process comprising
(a) measuring a level of a particulate in a process stream of the hydrocarbon refining process in communication with a hydrocarbon refinery component;
(b) identifying an effective amount of additive capable of reducing particulate-induced fouling based at least in part on the measured level of a particulate in the process stream and further based at least in part on the relative fouling potential of a crude oil present in the process stream; and
(c) introducing the effective amount of additive to the hydrocarbon refining process;
wherein the predetermined propensity for the process stream to foul is determined by:
(d) obtaining a first measurement of a characteristic indicative of an amount of fouling caused by the crude oil in the absence of any measurable particulate;
(e) obtaining a second measurement of the characteristic indicative of an amount of fouling caused by the crude oil in the presence of a predetermined amount of particulate;
(f) comparing the first measurement and the second measurement to identify the relative fouling potential of the crude oil.

17. An additive control system for controlling fouling in a hydrocarbon refining system comprising:
(a) a source of additive capable of reducing particulate-induced fouling in a hydrocarbon refining system;
(b) a valve to introduce to a process stream of the hydrocarbon refining system the additive capable of reducing particulate-induced fouling;
(c) a measuring device to measure a level of particulate in the process stream of the hydrocarbon refining system;
(d) a controller to control an amount of additive introduced into the process stream via the valve based upon the level of particulate measured in the process stream.

18. The system of claim 17, wherein the level of particulate measured in the process stream occurs in real-time.

19. The system of claim 17, wherein the level of particulate measured in the process stream is based on measurements obtained over a predetermined period of time.

20. The system of claim 17, wherein the controller receives input based on relative fouling potential of a crude oil present in the process stream.

21. The system of claim 17, wherein the particulate measuring device incorporates a microscope and particulate identification algorithms.

22. A method of determining the relative fouling potential of a crude oil comprising:
(a) obtaining a first measurement of a characteristic indicative of an amount of fouling caused by the crude oil in the absence of any measurable particulate;
(b) obtaining a second measurement of the characteristic indicative of an amount of fouling caused by the crude oil in the presence of a predetermined amount of particulate;
(c) comparing the first measurement and the second measurement to identify the relative fouling potential of the crude oil.

23. The method of claim 22, wherein the first amount measurement and second measurement are normalized based on the heat transfer ability of the crude oil blend.

24. The method of claim 22, wherein the process is repeated for at least two distinct crude oils, and the relative fouling potential in step (c) for the first crude oil blend is compared to the relative fouling potential obtained in step (c) for the second crude oil blend.

25. The method of claim 22, wherein the relative fouling potential is used for selecting the crude oil to be used in a hydrocarbon refining process.

26. The method of claim 22, wherein an amount of an additive capable of reducing particulate-induced fouling is identified based at least in part on the relative fouling potential of the crude oil.

27. The method of claim 26, wherein the amount of additive is further identified based at least in part on a real time measurement of the amount of the particulate in a process stream of a hydrocarbon refining process.

28. The method of 26 wherein the characteristic indicative of an amount of fouling is measured on a Alcor Hot Liquid Process Simulator.

29. The method of claim 22 wherein the type of additive is selected based on the relative fouling potential of the crude oil.
